(19) United States
(12) Patent Application Publication
(10) Pub. No.: US 2025/0354216 A1
(43) Pub. Date: Nov. 20, 2025

(54) METHODS AND SYSTEMS FOR MOLECULAR SUBTYPING OF CANCER METASTASES

(71) Applicant: THE UNIVERSITY OF CHICAGO, Chicago, IL (US)

(72) Inventors: Sean PITRODA, Chicago, IL (US); Ralph WEICHSELBAUM, Chicago, IL (US)

(73) Assignee: THE UNIVERSITY OF CHICAGO, Chicago, IL (US)

(21) Appl. No.: 18/867,289

(22) PCT Filed: May 18, 2023
(86) PCT No.: PCT/US2023/067189

§ 371 Date: Nov. 19, 2024

Related U.S. Application Data
(60) Provisional application No. 63/343,836, filed on May 19, 2022.

(51) Int. Cl. C12Q 1/6886; G16B 40/00; G16H 50/20

(52) U.S. Cl. CPC C12Q 1/6886; G16B 40/00; G16H 50/20; C12Q 2600/112; C12Q 2600/118; C12Q 2600/158

(57) ABSTRACT

Methods, assays, and compositions for identifying molecular subtypes of metastatic cancer are disclosed. The disclosed methods include determining expression levels of genes in a sample of metastatic tissue and identifying the molecular subtype of the metastasis based on the determined expression levels using a neural network-based classifier. Methods may further include providing a prognosis and making a treatment decision based on the molecular subtype of the metastasis. Further disclosed are methods of treatment of a cancer subject with a particular cancer therapy (e.g., local therapy, immunotherapy, EGFR inhibitor therapy) based on a molecular subtype of a metastasis from the subject.

### CROSS-REFERENCE TO RELATED APPLICATIONS

This application claims the benefit of priority to U.S. Provisional Patent Application Ser. No. 63/343,836, filed May 19, 2022, hereby incorporated by reference in its entirety.

### BACKGROUND OF THE INVENTION

### I. Field of the Invention

This invention relates generally to at least the fields of molecular biology and medicine.

### II. Background

Metastases are the leading cause of cancer-related deaths and are frequently widely disseminated, which has led to the prevailing view that metastases are always widespread. The oligometastasis hypothesis, in contrast, suggests that metastatic spread is a spectrum of virulence where some metastases are limited both in number and organ involvement and potentially curable with surgical resection or other loco-regional therapies^{1,2}. This paradigm is in stark contrast to the outcomes of patients with solid tumors where widespread metastases are largely fatal despite recent advances in systemic therapy. To date, the oligometastasis concept has been challenged, in large part, due to the lack of supporting molecular data to identify metastases associated with restricted spread^{3,4}.

Limited metastasis is relatively common. Data from clinical trials and single institution analyses of lung, breast, colorectal, prostate and renal cancers suggest that as many as 40-60% of patients with metastasis present with or develop limited disease⁵⁻⁵. Patients with limited liver metastases from colorectal cancer (CRC) have been consistently demonstrated to achieve prolonged survival after hepatic resection^{9,10} and provide an opportunity to investigate the molecular basis for oligometastasis. While there have been extensive investigations into the molecular subtypes of primary human cancers, little is known regarding molecular subtypes of metastasis and their relation to clinical outcomes.

There exists a need for robust, externally validated methods of identifying molecular subtypes of metastatic cancer, including CRC, that are predictive of clinical outcome and that can inform treatment decisions and prognosis.

### SUMMARY

Aspects of the present disclosure provide a validated classification process that identifies molecular subtypes of cancer metastases and informs treatment decisions, meeting various needs in the field of cancer medicine. Disclosed herein are methods comprising molecular classification of metastatic tissue to identify curable metastatic cancer and otherwise guide treatment decisions. As described herein, using a multi-layer neural network analysis of gene expression data in metastatic tissue samples expression signatures are identified that reliably classify metastatic samples into one of three subtypes—canonical, immune, and stromal—which correlate with different clinical outcomes and different treatment indications. The three subtypes correlate with different clinical outcomes, and knowing the subtype of the metastasis informs treatment decisions and helps provide an accurate assessment of patient prognosis. This discovery applies in metastatic cancers beyond only colorectal liver cancer—methods disclosed herein can be used to identify molecular subtypes of other metastatic cancers and to guide prognosis and treatment decisions for patients having such cancers.

Aspects of the present disclosure include methods for analyzing a tissue sample, methods for metastasis analysis, methods for gene expression analysis, methods for detecting differential gene expression in a tissue sample, methods for classifying a metastasis, methods for identifying a canonical subtype metastasis, methods for identifying an immune subtype metastasis, methods for identifying a stromal subtype metastasis, methods for methods for cancer diagnosis, methods for cancer prognosis, and methods for treating metastatic cancer. Methods of the present disclosure can include at least 1, 2, 3, 4, 5, or more of the following steps: collecting a tissue sample, collecting a metastasis sample, collecting a biological sample, extracting tumor RNA, performing RNA sequencing, performing a microarray analysis, measuring gene expression levels, measuring expression levels of one or more genes of Table 1, measuring expression levels of all the genes of Table 1, analyzing gene expression levels using a multi-layer neural network classification process, classifying a metastasis, administering a cancer therapy, administering a local therapy, administering an immunotherapy, and administering an EGFR inhibitor. Any one or more of the preceding steps may be excluded from certain aspects. Also disclosed, in some aspects, is a multi-layer neural classification system. A multi-layer neural classification system of the disclosure may comprise one or more of: an input layer, one or more hidden layers, and an output layer.

Disclosed herein, in some aspects, is a method of analyzing a tissue sample comprising measuring expression levels of one or more genes listed in Table 1 in a sample comprising tissue from a metastasis from a primary cancer tumor. Expression levels of any one or more of the genes listed in Table 1 may be measured and/or analyzed in a method of the disclosure, including any and all combinations of the genes listed in Table 1. In some aspects, expression levels of at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92,93,94,95,96,97,98,99, 100,101,102,103,104, 105, 106,107,108,109,110,111,112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 of the genes of Table 1. In some aspects, expression levels of LAYN, RNF150, MICU3, CAMK4, TM6SF1, MAPK10, SLC16A2, NEXN, SSPN, PCDH9, TLR6, PCDH18, HDAC9, ABCA6, RASSF8, EPHA3, ITGBL1, TEK, ST3GAL6, KCNE4, CARD6, JAML, PREX2, PLEKHH2, CEP85L, RHOJ, DZIP1, IL7R, MGP, MRC1,CYRIA, PIK3CG, GUCY1B1, FAP, GNG2, MITF, FRMD6, PLAT, MSRB3, LUM, GAS2L1, LDB2, CPQ, GLIPR1, LRRC8C, RNF144B, S1PR3, CLCN2, CDH11, FYB1, SDC2, ANTXR1, MEF2C, ALDH16A1, MAF, HCFC2, MARCHF2, HMCN1, ZNF865, RNF166, GPR137, ZNF654, PTPRM, TSSC4, IGFBP7, QKI, ANKRD49, TELO2, CRIPT, TCIRG1, PKD2, ETS1, SCOC, GOLT1B, PIGF, CCDC9, LCORL, UFL1, ELMOD2, SCAF1, DHX40, CARNMT1, NFYB, IL6ST, ERF, SNRNP48, IKZF5, CFAP97, MIGA1, RARS2, SPAST, ABCE1, COPS2, PIK3CA, NPAT, RBAK, NOB1, C2or f49, ATAD1, DCAF17, PPP1R12C, PUS7L, FRMD8, CEBPZ, EML3, RICTOR, PPP1R9B, PPP6C, KDM6B, LIN7C, NUDT21, ZNF326, SEPTIN7, PREPL, ZNF507, NUCB1, FXR1, MARCHF7, U2SURP, HNRNPH3, TYK2, CREB1, PHIP, HNRNPA1, RYK, TLK1, STAG1, FBXO11, PAPOLA, RBM12, FUBP1, ATRX, PIK3C2A, RSF1, PRPF4B, IP08, SENP6, CCNT1, MFF, ZNF638, EIF4A2, NIPBL, USP34, MARCHF6, EIF3B, MOBlA, INO80D, RBMX, RC3H1, and/or HNRNPA2B1, including any combination thereof. In some aspects, no expression levels of genes are measured other than those listed in Table 1. It is also contemplated that one or more of the genes listed in Table 1 may be excluded. In some aspects, the expression levels of the one or more genes are within a predetermined amount of a mean expression level in metastases of a cohort of patients having one of the following three metastatic phenotypes: canonical, immune, or stromal.

Disclosed herein, in some aspects, is a method of analyzing a tissue sample comprising measuring expression levels of all of the genes of Table 1 in a sample comprising tissue from a metastasis from a primary cancer tumor.

In some aspects, the method further comprises calculating a clinical risk score for the patient. In some aspects, the method further comprises analyzing the expression levels of the one or more genes using a multi-layer neural network classification process that includes an input layer, one or more hidden layers, and an output layer. In some aspects, the input layer comprises the expression levels of the one or more genes of Table 1. In some aspects, the output layer comprises a classification of the expression level data of the input layer as indicating a canonical, an immune, or a stromal metastatic phenotype. In some aspects, the classification process comprises determining the probability that the metastasis has a canonical, immune, or stromal metastatic phenotype. In some aspects, the classification process comprises determining each of the three probabilities of the metastasis having a canonical, immune, and metastatic phenotype. In some aspects, the neural network classification process comprises a first hidden layer and a second hidden layer.

In some aspects, the method further comprises, prior to measuring the expression levels, obtaining the sample from a subject. In some aspects, the sample is from a subject. In some aspects, the method further comprises administering a cancer therapy to the subject. In some aspects, the cancer therapy comprises a local cancer therapy and does not comprise a systemic cancer therapy. In some aspects, the cancer therapy comprises an immunotherapy.

In some aspects, measuring the expression levels of the one or more genes comprises RNA sequencing. In some aspects, measuring the expression levels of the one or more genes comprises a microarray. In some aspects, measuring the expression levels of the one or more genes comprises performing polymerase chain reaction.

In some aspects, the primary cancer tumor is a colorectal cancer tumor. In some aspects, the primary cancer tumor is not a colorectal cancer tumor (e.g., is a liver cancer, testicular cancer, biliary cancer, ovarian cancer, urinary tract cancer, pancreatic cancer, prostate cancer, esophageal cancer, gastric cancer, head and neck cancer, cervical cancer, lung cancer, neuroendocrine cancer, kidney cancer, breast cancer, or melanoma tumor). In some aspects, the metastasis is a liver metastasis.

Also disclosed herein, in some aspects, is a method of treating metastatic cancer in a patient, the method comprising administering to the patient a local cancer therapy without administering systemic cancer therapy, administering to the patient an immunotherapy, or administering to the patient an EGFR inhibitor, wherein the patient has been determined to have a metastasis having expression levels of one or more genes listed in Table 1 that indicate a canonical or immune metastatic phenotype based on a multi-layer neural network classification process. In some aspects, the input layer comprises the expression levels of the one or more genes of Table 1. In some aspects, the input layer comprises the expression levels of all of the genes of Table 1. In some aspects, the output layer comprises a classification of the expression level data of the input layer as indicating a canonical, an immune, or a stromal metastatic phenotype.

Further disclosed, in some aspects, is a method of treating metastatic cancer in a patient, the method comprising administering to the patient a local cancer therapy without administering systemic cancer therapy or administering to the patient an immunotherapy or EGFR inhibitor, wherein the patient has been determined to have a metastasis having expression levels of one or more genes listed in Table 1 (e.g., two or more or all of the genes listed in Table 1) that are within a predetermined amount of the mean expression level of the one or more genes in metastases of a cohort of metastatic cancer patients having a mean overall five-year survival expectation that is at least 60% or a mean five-year disease-free survival expectation that is at least 30%. In some aspects, the expression levels of the one or more genes indicate a canonical or immune metastatic phenotype. In some aspects, an expression signature of the one or more genes matches an expression signature of a canonical or immune metastatic phenotype. In some aspects, the expression levels of the one or more genes have been used as an input layer of a multi-layer neural network classification system.

Also disclosed herein, in some aspects, is a method of treating cancer in a patient having a metastasis from a primary cancer tumor, the method comprising: administering to the patient an immune checkpoint therapy or administering to the patient a local cancer therapy without administering a systemic cancer therapy, wherein the patient has been identified based on expression levels of one or more genes in the metastasis as belonging to a group of metastatic cancer patients with one or more of the following characteristics: (a) a mean five-year overall survival expectation of at least 60%; (b) a mean five-year disease-free survival expectation of at least 30%; (c) a likelihood of experiencing metastatic recurrence after hepatic resection that is lower than the likelihood for patients outside of the group; (d) a canonical metastatic phenotype; and (e) an immune metastatic phenotype.

Further disclosed, in some aspects, is a method of diagnosing a patient having a metastasis from a primary colorectal cancer tumor, the method comprising: (a) determining expression levels in the metastasis of one or more of the genes (e.g., two or more genes or all of the genes) listed in Table 1; and (b) identifying the patient as having a canonical metastatic phenotype, as having an immune metastatic phenotype, as being a responder to immune checkpoint cancer therapy, as having a five-year overall survival expectation of greater than 60%, or as having a five-year disease-free survival expectation of greater than 30% if the expression level of one or more of the genes is within a predetermined amount of a first reference expression level or deviates from a second reference expression level by a predetermined amount. It is also contemplated that one or more of the genes listed in Table 1 may be excluded. In some aspects, the first reference expression level represents the mean expression level in metastases of a cohort of metastatic cancer patients having a canonical metastatic phenotype, having an immune metastatic phenotype, being a responders to immune checkpoint cancer therapy, having a five-year overall survival expectation of greater than 60%, and/or having a five-year disease-free survival expectation of greater than 30%. In some aspects, the second reference expression level represents the mean expression level in metastases of a cohort of metastatic cancer patients having a mean five-year overall survival expectation of less than 60%.

Additionally disclosed, in some aspects, is a method of treating a patient having a metastasis from a primary colorectal cancer tumor, the method comprising: (a) measuring the expression of one or more genes in a sample from the metastasis; comparing the measured expression level of each gene to a reference expression level for that gene; identifying the metastasis as having a canonical, immune, or stromal phenotype based on the measured expression levels; and administering to the patient an appropriate therapy based on the type of metastasis identified in step (c). In some aspects, the method comprises measuring the expression level of at least 1, 2, 3, 4, 5, 10, 20, 50, 100, or all of the genes of Table 1. It is also contemplated that one or more of the genes listed in Table 1 may be excluded. In some aspects, (b) comprises analyzing the expression level of each gene using a multi-layer neural network classification system having an input layer, one or more hidden layers, and an output layer, wherein the input layer comprises the expression levels of the one or more genes and wherein the output layer comprises a classification of the expression level data of the input layer as indicating a canonical, an immune, or a stromal metastatic phenotype.

If the expression levels of the genes measured in a sample metastasis are sufficiently close to the reference expression levels of a metastatic subtype, then the sample metastasis can be classified as being of that subtype. The degree of closeness in expression levels required to be classified as a match may be predetermined using a statistical analysis, including a neural network classification process. In some embodiments, the predetermined amount of closeness is within one standard deviation of the mean expression level of the reference cohort. In some embodiments, the predetermined amount is within 0.1, 0.5, 1.0, 2.0, 3.0, 4.0, 5.0, 10, 15, or 20% of the reference expression level, or any range derivable therein. In some embodiments, a sample metastasis may be classified as belonging to a molecular subtype despite the expression levels of one or more genes deviating from a reference expression level by a substantial amount. For instance, if a substantial number of other gene expression levels sufficiently match the reference expression, then the sample metastasis may be classified as belonging to the subtype. A computer-based classifier programmed to perform a statistical analysis may be used to determine whether expression levels of a sufficient number of genes in a sample metastasis are sufficiently close to the reference expression levels of a particular molecular subtype to classify the sample as belonging to that subtype. The computer-based classifier program may comprise a neural network classification process or may have been derived using a neural network process.

In some aspects, the appropriate therapy for a patient with a canonical-type metastasis comprises a DNA damaging chemotherapy, PARP inhibitor, angiogenesis inhibitor, and/or MYC inhibitor. In some aspects, the appropriate therapy for a patient with an immune-type metastasis comprises an EGFR inhibitor, immunotherapy, and/or a splicing inhibitor. In some aspects, the appropriate therapy for a patient with a stromal-type metastasis comprises an angiogenesis inhibitor, KRAS inhibitor, and/or tumor stromal inhibitor, or excludes an EGFR inhibitor. Any of the therapies may be specifically excluded.

Also disclosed, in some aspects, is a method of treating a patient having metastatic colorectal cancer, the method comprising administering an EGFR inhibitor to a patient who has been tested and found to have liver metastases of an immune molecular subtype by analyzing the expression levels of transcripts of at least two of the genes (e.g., all of the genes) listed in Table 1. It is also contemplated that one or more of the genes listed in Table 1 may be excluded. In some aspects, the expression levels of the genes are analyzed using a neural network classification process. In some aspects, the input into the neural network classification process consists of all the genes listed in Table 1. In some aspects, the input into the neural network classification process includes only genes listed in Table 1. It is also contemplated that one or more of the genes listed in Table 1 may be excluded. In some aspects, the EGFR inhibitor is cetuximab. In some aspects, the EGFR inhibitor is panitumumab.

Further disclosed is a method of diagnosing a patient having a liver metastasis from a primary colorectal cancer tumor, the method comprising inputting the expression levels in the metastasis of one or more of the genes listed on Table 1 into a classifier that has been trained to recognize an expression signature of a canonical, immune, and/or stromal metastatic molecular subtype. In some aspects, the classifier has been trained using a neural network machine learning process. In some aspects, the expression levels of all the genes listed on Table 1 are inputted into the classifier. In some aspects, no other expression levels are inputted into the classifier. It is also contemplated that one or more of the genes listed in Table 1 may be excluded.

In some embodiments, gene expression measurement and analysis of the present disclosure may indicate that one or more cancer therapies would be likely to be effective or ineffective. A particular advantage of methods disclosed herein is that they allow medical providers to make a treatment decision based on the molecular subtype of a metastasis. The discoveries disclosed herein indicate that some metastatic subtypes, such as immune, for example, are more likely to respond to a local therapy such as resection, radiation therapy, and the like, without the need for a systemic cancer therapy. The discoveries disclosed herein also allow medical providers to identify metastatic cancer for which a local therapy may not be helpful and/or for which systemic therapies, such as DNA damaging drugs, are appropriate.

In any of the embodiments described herein, gene expression analysis can be performed using a classifier that was trained using a neural network process having as inputs at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100,101,102,103,104,105,106, 107,108,109,110,111,112,113,114,115,116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 of the genes listed in Table 1, or any range derivable therein. It is also contemplated that one or more of the genes listed in Table 1 may be excluded. In some embodiments, the trained classifier assigns a probability that a given set of expression levels represents an expression signature of a canonical, immune, or stromal molecular subtype. In some embodiments, the expression signatures were previously determined by a neural network classification process. In some embodiments, the trained classifier compares input expression levels of the genes to reference expression levels of the genes, wherein the reference expression levels were determined using a neural network classification process. In some embodiments, the trained classifier compares input expression levels of the genes to reference expression signatures for canonical, immune, and/or stromal metastatic subtypes.

In any of the embodiments described herein, the patient may have already been diagnosed with cancer or already had tumor resection before any of the steps of methods described herein are performed.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a neural network classification process.

FIGS. 2A and 2B show a comparison of the molecular subtypes of the CRCLM samples in the UK study cohort.

FIGS. 3A and 3B show disease-free survival (FIG. 3A) and overall survival (FIG. 3B) for patients from the UK cohort in the low/intermediate risk vs. high risk groups.

FIGS. 4A and 4B show additional details of the UK cohort patients. FIG. 4A shows subtype of patients in the different treatment arms. FIG. 4B shows KRAS signaling in each of the molecular subtypes.

FIG. 5 shows disease-free survival Kaplan-Meier curves for the three molecular subtypes in the two treatment arms in the UK study.

### DETAILED DESCRIPTION

Aspects of the present disclosure are based, at least in part, on the development of a fully validated 150 mRNA-based molecular signature which classifies patients with metastatic colorectal cancer to the liver into one of three prognostic molecular subtypes. In certain aspects, such a molecular signature can personalize potentially curable treatment approaches for patients with metastatic colorectal cancer. Accordingly, aspects of the present disclosure are directed to methods and systems for measuring expression levels of one or more genes of Table 1 from a metastasis from a tumor. Also described are methods for classification of metastatic cancer in a patient based on expression levels of one or more genes of Table 1 from a metastasis. Further disclosed are methods for treatment of metastatic cancer based on classification of the cancer based on expression levels of one or more genes of Table 1.

### I. Gene Expression Levels

Methods disclosed herein include measuring expression of genes. Measurement of expression can be done by a number of processes known in the art. The process of measuring expression may begin by extracting RNA from a metastasis tissue sample. Extracted mRNA can be detected by hybridization (for example by means of Northern blot analysis or DNA or RNA arrays (microarrays) after converting mRNA into labeled cDNA), by amplification by means of an enzymatic chain reaction, or any other detection methods recognized in the art. Quantitative or semi-quantitative enzymatic amplification methods such as polymerase chain reaction (PCR) or quantitative real-time RT-PCR or semi-quantitative RT-PCR techniques can be used. Primer pairs may be designed for the purpose of superimposing an intron to distinguish cDNA amplification from the contamination from genomic DNA (gDNA). Additional primers or probes, which may be labeled, for example with fluorescence, which hybridize specifically in regions located between two exons, are optionally designed for the purpose of distinguishing cDNA amplification from the contamination from gDNA. If desired, said primers can be designed such that approximately the nucleotides comprised from the 5′ end to half the total length of the primer hybridize with one of the exons of interest, and approximately the nucleotides comprised from the 3′ end to half the total length of said primer hybridize with the other exon of interest. Suitable primers can be readily designed by a person skilled in the art. Other amplification methods include ligase chain reaction (LCR), transcription-mediated amplification (TMA), strand displacement amplification (SDA) and nucleic acid sequence based amplification (NASBA). Expression levels of genes may also be measured by RNA sequencing methods known in the art.

To normalize the expression values of one gene among different samples, comparing the mRNA level (also “expression level”) of the gene of interest in the samples from the subject object of study with a control RNA level is possible. As it is used herein, a “control RNA” is an RNA of a gene for which the expression level does not differ among different metastatic subtypes, for example a gene that is constitutively expressed in all types of cells. A control RNA is preferably an mRNA derived from a housekeeping gene encoding a protein that is constitutively expressed and carrying out essential cell functions.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. For example, the reference expression level for a gene can be based on the mean expression level of the gene obtained from a number of patients who have immune subtype metastases. A reference level can be based on the expression levels of the markers to be compared obtained from samples from subjects who do not have a disease state or a particular phenotype. The person skilled in the art will see that the particular reference expression level can vary depending on the specific method to be performed.

Some embodiments include determining that a measured expression level is higher than, lower than, increased relative to, decreased relative to, equal to, or within a predetermined amount of a reference expression level. In some embodiments, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain gene at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600,700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. This can be the case, for example, when a classifier is used to identify the molecular subtype of a metastasis. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene expression levels to a mean expression levels or a reference expression levels, the comparison is to be made on a gene-by-gene basis. For example, if the expression levels of gene A and gene B in a patient's metastasis are measured, a comparison to mean expression levels in metastases of a cohort of patients would involve: comparing the expression level of gene A in the patient's metastasis with the mean expression level of gene A in metastases of the cohort of patients and comparing the expression level of gene B in the patient's metastasis with the mean expression level of gene B in metastases of the cohort of patients. Comparisons that involve determining whether the expression level measured in a patient's metastasis is within a predetermined amount of a mean expression level or reference expression level are similarly done on a gene-by-gene basis, as applicable.

### II. Identifying Molecular Subtypes of Metastases

Methods disclosed herein can be used to identify different molecular subtypes of metastatic cancer that correlate with different clinical outcomes and different sensitivities to particular treatment regimens. The subtypes can be identified using a multi-layer neural network classification technique.

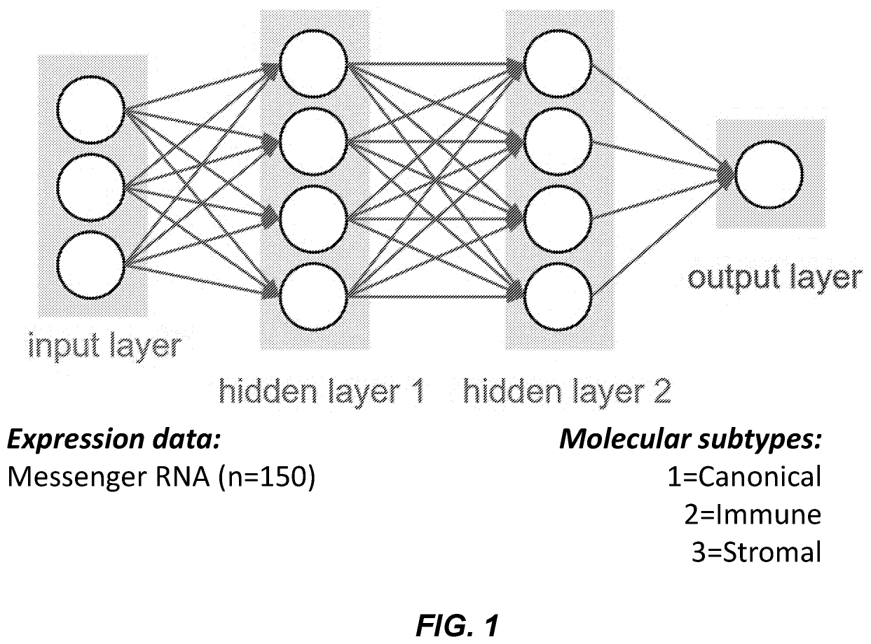

A neural network is a machine learning computing system that consists of a number of simple but highly interconnected elements or nodes, called ‘neurons’, which are organized in layers which process information using dynamic state responses to external inputs. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks. Neural network systems are useful in finding expression signatures that are too complex to be manually derived and taught to a machine. A neural network can be constructed for a selected set of expression levels. In multilayer neural networks, there are input units (input layer), hidden units (hidden layer), and output units (output layer); a diagram of an example multilayer neural network is shown in FIG. 1. There is, furthermore, a single bias unit that is connected to each unit other than the input units. Neural networks are described in, for example, Duda et al., 2001, Pattern Classification, Second Edition, John Wiley & Sons, Inc., New York; and Hastie et al., 2001, The Elements of Statistical Learning, Springer-Verlag, New York, each of which is incorporated herein by reference in its entirety.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

### III. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy, skin biopsy, and liquid biopsy. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. In some aspects, the biological sample is a cell-free sample. In some aspects, the biological sample is a sample comprising cell-free DNA (cfDNA), for example circulating tumor DNA (ctDNA). The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen, blood collection, or plasma collection.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple lung samples may be obtained for diagnosis by the methods described herein. In other cases, multiple samples, such as one or more samples from one tissue type (for example lung) and one or more samples from another specimen (for example serum) may be obtained for diagnosis by the methods. In some cases, multiple samples such as one or more samples from one tissue type (e.g. lung) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a plasma sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a lung or a suspected lung tumor or neoplasm. In one embodiment, the sample is a fine needle aspirate of a lung or a suspected lung metastasis of a primary tumor (e.g., colorectal cancer tumor). In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Kits

Certain aspects of the present disclosure also concern kits containing compositions of the disclosure or compositions to implement methods disclosed herein. In some embodiments, kits can be used to evaluate one or more biomarkers (e.g., 1, 2, 3, 4, 5, 10, 20, 50, or 150 of the genes of Table 1). In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, non-synthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

### V. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 75 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### B. RNA Sequencing

In some embodiments, RNA may be analyzed by sequencing. The RNA may be prepared for sequencing by any method known in the art, such as poly-A selection, cDNA synthesis, stranded or non-stranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

### VI. Cancer Therapy

In some embodiments, the disclosed methods comprise administering a cancer therapy to a subject or patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises a radiotherapy. In some embodiments, the cancer therapy comprises a chemotherapy. In some embodiments, the cancer therapy comprises an immunotherapy, which may be a checkpoint inhibitor therapy. Any of these cancer therapies may also be excluded. Combinations of these therapies may also be administered.

Methods disclosed herein may include administering a cancer therapy or determining a course of cancer treatment based on an identified metastatic subtype. Some embodiments include administering a local cancer treatment or determining that a local cancer treatment is appropriate. Local cancer treatments include those that target cancer tissue using a technique directed to a specific organ or limited area of the body. Local cancer treatments include surgery (i.e., resection), radiation therapy, cryotherapy, laser therapy, topical therapy, high intensity focused ultrasound, and photodynamic therapy. A local treatment (also “local therapy”) may include stereotactic body radiotherapy (SBRT), stereotactic ablative body radiotherapy (SABR), stereotactic radiosurgery (SRS), radiofrequency ablation (RFA), percutaneous cryoablation therapy (PCT), and photodynamic therapy (PDT). A local therapy may be directed at the primary tumor and/or at one or more metastases.

Systemic cancer therapies are those that are distributed widely within the body, such as a variety of drug treatments, which may be delivered orally or intravenously. Examples of systemic therapies include chemotherapy, hormone therapy, immunotherapy, and targeted therapy (i.e., drugs that are distributed widely within the body, but have targeted effects on cancer cells).

Identifying the molecular subtype of metastatic colorectal cancer can be used to determine an appropriate treatment regimen. In some embodiments, the appropriate treatment for canonical subtype metastases include EGFR inhibitors (e.g., anti-EGFR antibodies such as cetuximab and panitumumab; small molecule EGFR inhibitors such as erlotinib, afatinib, gefitinib, lapatinib, and osimertinib; etc.); PARP inhibitors; PI3K inhibitors; NOTCH inhibitors; angiogenesis inhibitors; DNA damaging agents such as cisplatin, oxaliplatin, carboplatin, cyclophosphamide, chlorambucil, or temozolomide; STING agonists; innate immune agonists; RNA vaccines; MYC inhibitors; or combinations thereof. In some embodiments, the appropriate treatment for immune subtype metastases include EGFR inhibitors, PD-1/PD-L1 immunotherapies, other immunotherapies, beta-secretase inhibitors, lipid-lowering agents, splicing inhibitors, and combinations thereof. In some embodiments, the appropriate treatment for stromal subtype metastases include PDGF/PDGFR inhibitors, KRAS inhibitors, tumor stromal inhibitors, VEGF/VEGFR inhibitors, angiogenesis inhibitors, JAK1/JAK2 inhibitors, COX2 inhibitors, HDAC inhibitors, DNA demethylating agents, other epigenetic modifiers, and combinations thereof. In some embodiments, the appropriate treatment for stromal subtype metastases excludes an EGFR.

In some embodiments, methods herein include administering cetuximab, a monoclonal antibody that binds epidermal growth factor receptor (EGFR), to patients depending on the molecular subtype of their metastases. In some embodiments, cetuximab is administered to patients who have been tested and determined to have immune molecular subtype metastases. In some embodiments, the cetuximab is administered weekly or every other week. In some embodiments, an initial dose of 400 mg/m2 is administered, followed by weekly doses of 250 mg/m2. In some embodiments, the initial dose is at least about, at most about, or about 100, 150, 200, 250, 300, 350, 400, 450, or 500 mg/m2, or is between any two of these values. In some embodiments, the subsequent weekly doses are at least about, at most about, or about 50, 100, 150, 200, 250, 300, 350, or 400 mg/m2, or are between any two of these values. The doses may be infused over the course of 1 to 2 hours at an infusion rate of no more than 10 mg/min. In some embodiments, the patient is tested and determined to have a KRAS wild type genotype.

In some embodiments, panitumumab, an EGFR receptor-binding monoclonal antibody, is administered to a patient who has immune molecular subtype metastases. In some embodiments, the dosage administered is 6 mg/kg every other week. In some embodiments, the dosage is at least about, at most about, or about 1, 2, 3, 4, 5, 6, 7, 8, 9, or 10 mg/kg every other week, or is between any two of these values.

Methods disclosed herein can also include making treatment decisions based on an integrated risk group classification of a patient. This classification combines the molecular subtyping of the metastasis with a clinical risk score of the patient and divides patients into low risk, intermediate risk, and high risk groups based on their respective five-year probabilities of disease-free survival or overall survival. A patient's integrated risk group indicates the likelihood of benefit from local metastasis-directed therapies such as surgical resection, stereotactic body radiotherapy (SBRT), stereotactic ablative body radiotherapy (SABR), stereotactic radiosurgery (SRS), radiofrequency ablation (RFA), percutaneous cryoablation therapy (PCT), and photodynamic therapy (PDT): low-risk patients have the highest likelihood of benefit from these therapies, high-risk patients have the lowest likelihood of benefit from these therapies, and intermediate-risk patients have an intermediate likelihood of benefit from these therapies.

Conventionally, it has been thought that metastatic cancer always requires a systemic therapy. However, determination of the molecular subtypes of metastatic cancer as described herein can be used to indicate metastatic cancers, such as those with canonical or immune subtype metastases, are likely to respond favorably to local therapies and may not need an additional systemic therapy. Conversely, some metastatic cancers, such as those with stromal subtype metastases, are not likely to respond to local therapy alone, or at all, and should therefore be treated with appropriate systemic therapies.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is a Stage I cancer. In some embodiments, the cancer is a Stage II cancer. In some embodiments, the cancer is a Stage III cancer. In some embodiments, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, disclosed are methods for treating cancer originating from the colon. In some embodiments, the cancer is colon cancer. In some aspects, the cancer is colorectal cancer. In some embodiments, the cancer is metastatic cancer. In some aspects, the cancer is liver cancer, testicular cancer, biliary cancer, ovarian cancer, urinary tract cancer, pancreatic cancer, prostate cancer, esophageal cancer, gastric cancer, head and neck cancer, cervical cancer, lung cancer, neuroendocrine cancer, kidney cancer, breast cancer, or melanoma.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

In some embodiments, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some embodiments, the radiotherapy can comprise external radiotherapy, internal radiotherapy, radioimmunotherapy, or intraoperative radiation therapy (IORT). In some embodiments, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some embodiments, the internal radiotherapy comprises interstitial brachytherapy, intracavitary brachytherapy, or intraluminal radiation therapy. In some embodiments, the radiotherapy is administered to a primary tumor.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of radiotherapy administered to a subject may be presented as a total dose of radiotherapy, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### B. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

### a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDIO680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

### d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFN7-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another embodiment, the antibody has at least about 70, 75, 80, 85, 90, 95, 97, or 99% (or any derivable range or value therein) variable region amino acid sequence identity with the above-mentioned antibodies.

### 2. Activation of Co-Stimulatory Molecules

In some embodiments, the immunotherapy comprises an activator of a co-stimulatory molecule. In some embodiments, the activator comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein. For example, the patient may be one that has been determined to be sensitive to an immune checkpoint inhibitor therapy based on a determination that the patient has or previously had pancreatitis.

### C. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Clinical Characteristics and Patient Outcomes

The inventors previously identified three molecular subtypes of colorectal liver metastases (CRCLM) designated as canonical (SNF1), immune (SNF2), and stromal (SNF3) subtypes. See Pitroda et al., “Integrated molecular subtyping defines a curable oligometastatic state in colorectal liver metastasis,” Nature Communications 9:1793 (2018) (hereinafter, “Pitroda 2018 Publication”); WO2019/204576. The purpose of the current study was to develop an efficient classification process using fewer expression level inputs and to validate the existence of and prognostic differences between these three molecular subtypes in an independent clinical cohort.

Few retrospective cohorts of CRCLM are available, and those that are available are limited by small sample sizes and non-randomized selection of patients for analysis. Herein is presented a validation of the molecular subtypes in a randomized clinical trial from the UK using a unique neural network-based classifier. In this trial, patients received standard of care pre-operative/post-operative chemotherapy, including neoadjuvant plus adjuvant chemotherapy and complete resection of all CRCLM. Primary tumors were also treated with curative intent. The study included 257 KRAS wild-type (codons 12, 13, & 61) colorectal cancer patients, and 80% of the patients had 1 to 3 liver metastases. The randomization was to +/− cetuximab in the pre-operative and post-operative settings. Ultimately, the trial was negative for the primary endpoint. Microarray profiling of 147 CRCLM specimens for gene expression were performed using the Affymetrix Xcel microarray platform. Mutational and CNV analyses were also performed.

In the present study, the inventors aimed to minimize the number of input mRNA features as part of a machine learning classifier while maintaining a high accuracy for classification into the three molecular subtypes. The inventors first overlapped the mRNA features that were present in the Pitroda 2018 Publication with the data from the UK randomized trial Xcel platform. This provided the full set of potential input mRNA features. The inventors utilized a neural network classifier (a machine learning algorithm) to derive a classifier in the cohort from the Pitroda 2018 Publication that could then be validated in the UK validation cohort. In this context, 2018 study cohort was split into a training and testing set (60% and 40% of samples respectively) from which a signature was discovered and iteratively optimized. The model was first derived by training the neural network containing a hidden layer of 35 neurons and using as the input standardized z-scores of 500 mRNA expression values for each patient in the 2018 study cohort. The 500 mRNAs were selected from 17,162 mRNAs on the basis of having the highest principal components (PC1 and PC2) using a principal components analysis. At this initial stage the average model accuracy using 500 mRNAs as input features was 80% in the 2018 cohort testing set. In order to improve the model prediction, a recursive feature elimination was performed where input features that did not contribute significantly to the model accuracy were successively eliminated. The final model contained only 150 mRNAs (listed in Table 1 below).

The average model accuracy using the set of 150 features was 90% in the 2018 study testing cohort. Using randomly selected sets of 60% of the input data (UK cohort), 100 independent neural network models were generated to predict the three molecular subtypes. Each model provides an output for the probability that a given sample corresponds to the canonical, immune, and stromal subtypes. Using the set of 100 trained neural network models, a subtype classification was performed on the UK cohort where the final subtype for each sample corresponds to the most frequent subtype chosen by the 100 models. FIG. 1 shows a schematic of a neural network classification model. The input layer comprises input data such as mRNA expression data. The classification model can have multiple hidden layers, each with a number of nodes, or neurons. The output layer provides probabilities that the input data fits into one or more classes, such as one or more of the three molecular subtypes of CRCLM.

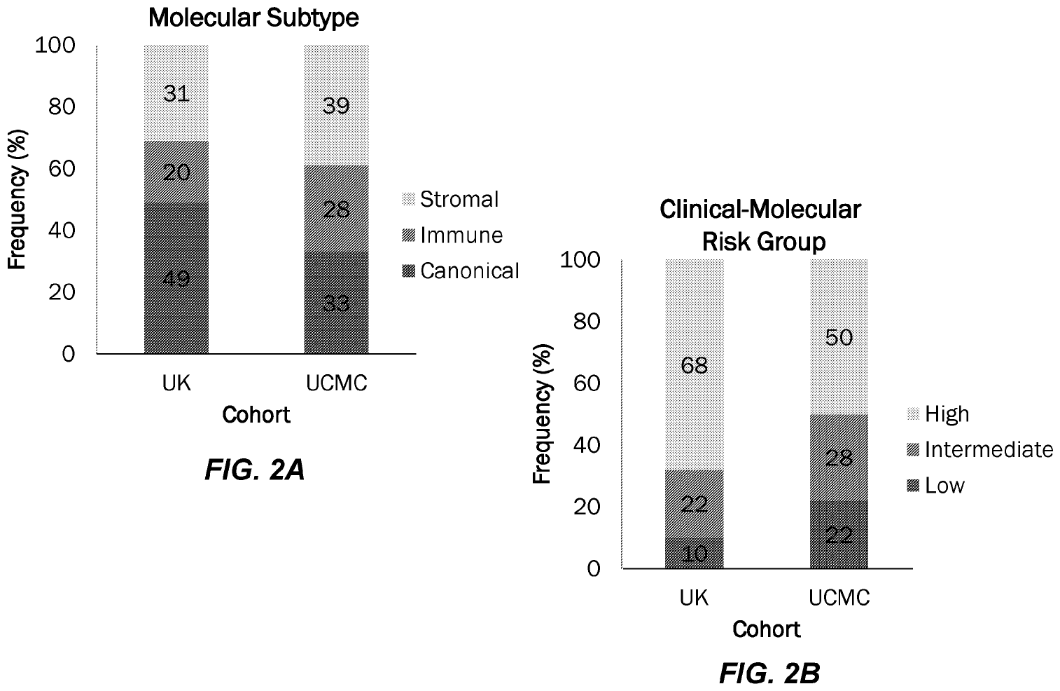

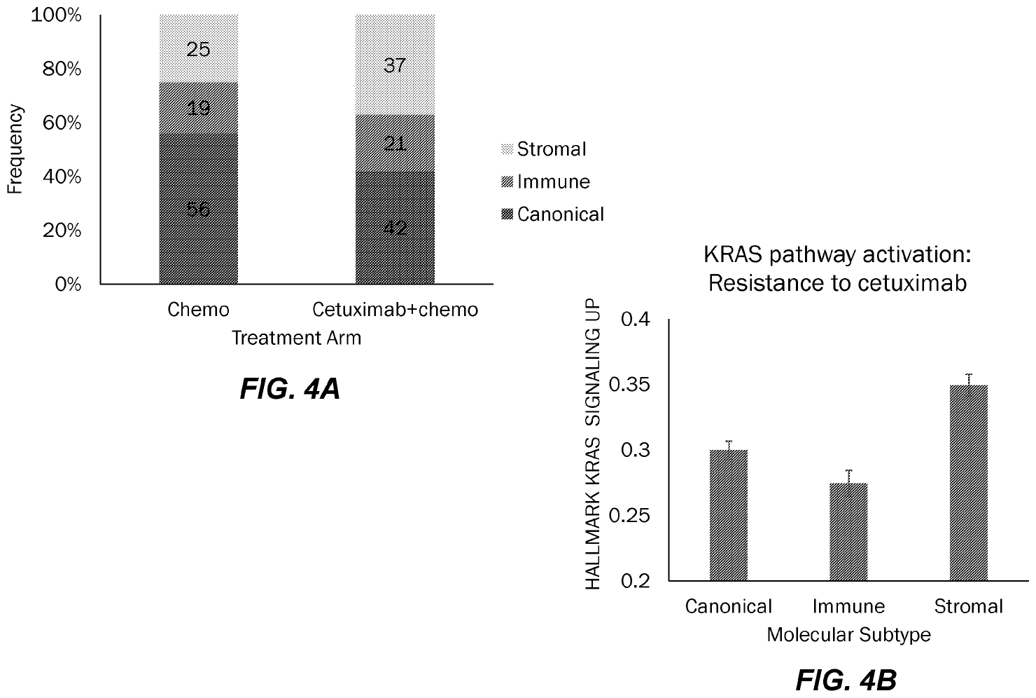

FIGS. 2A and 2B show a comparison of the molecular subtypes of the CRCLM samples in the UK study cohort (labeled “UK” in FIGS. 2A and 2B) and the Pitroda 2018 Publication study cohort (labeled “UCMC” in FIGS. 2A and 2B). The distribution of the CRCLM molecular subtypes is different across the UK and Pitroda 2018 Publication cohorts with greater frequencies of the adverse subtypes (canonical and stromal) in the UK cohort (FIG. 2A). Moreover, the inventors previously proposed an integrated risk classification based on molecular subtypes and clinical risk scores (Pitroda 2018 Publication, FIG. 4). The distribution of the integrated risk groups in the UK cohort was examined, and significantly fewer low risk patients and much higher frequency of high risk patients (i.e. patients who are likely to have poor clinical outcomes after treatment) were found (FIG. 2B).

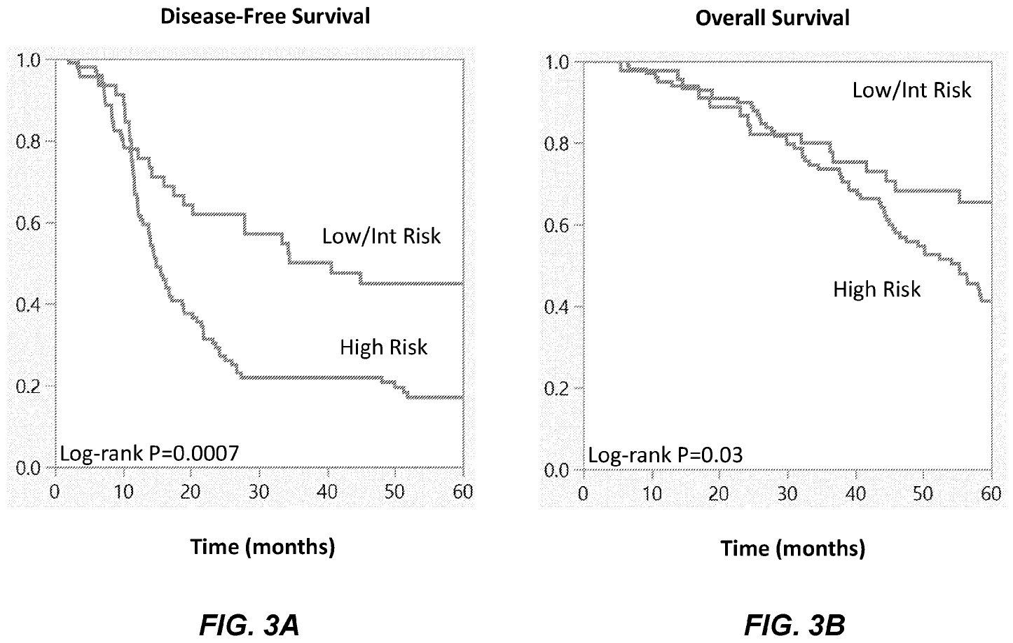

Importantly, patients in the UK cohort had significantly different disease free and overall survival based on the integrated risk group classification (FIG. 2B). FIG. 3 shows that patients in the low+intermediate risk group using the integrated risk group classification have nearly 25% (absolute) improvements in disease free and overall survivals as compared to high risk patients. This is a direct validation of the existence and prognostic impact of the molecular subtypes identified herein in a prospective clinical cohort.

Given that the UK trial was negative for its primary endpoint of a disease free survival benefit with the addition of cetuximab to standard chemotherapy, the inventors tested whether the CRCLM molecular subtypes could provide an explanation for their clinical outcomes. A statistical imbalance between the standard of care chemotherapy arm and the chemotherapy+cetuximab arm was found, with more stromal (adverse subtype) patients in the cetuximab arm (FIG. 4A). Moreover, the tumors exhibiting the stromal phenotype had increased KRAS signaling activation (FIG. 4B), which is a known resistance mechanism to cetuximab.

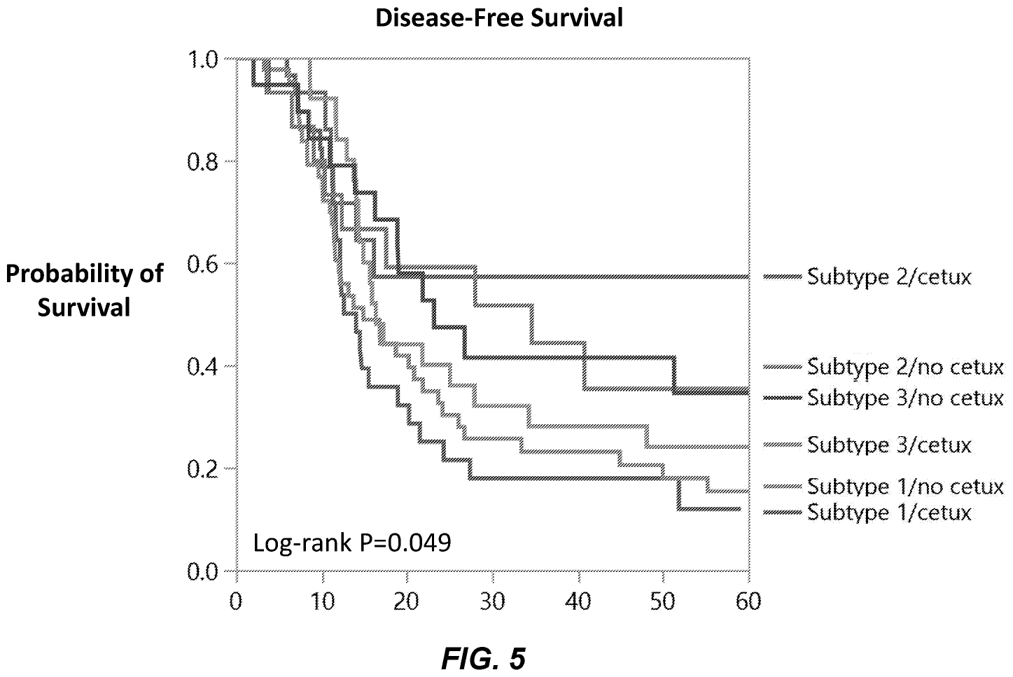

The inventors determined the disease-free survival Kaplan-Meier curves for the three molecular subtypes in the two treatment arms in the UK study (cetuximab+ or −) (see FIG. 5). Patients with CRCLM tumors of the canonical molecular subtype showed no difference in disease free survival with or without cetuximab. Patients with CRCLM tumors of the immune subtype had an improvement in disease free survival with cetuximab, indicating that cetuximab would be clinically useful for this subset of patients. By contrast, patients with CRCLM tumors of the stromal subtype had a detriment in disease-free survival with cetuximab. The patients treated with cetuximab were more likely to develop widespread recurrences after their initial treatment, which may be due to cetuximab treatment selecting pre-existing tumor clones or causing the emergence of drug resistant tumor clones due to elevated KRAS signaling in these tumors, leading to increased distant metastasis and death in patients with the stromal CRCLM subtype.

TABLE 1

| Alias | Gene Name | Description |
|---|---|---|
| ENSG00000204381 | LAYN | layilin [Source: HGNC Symbol; Acc: HGNC: 29471] |
| ENSG00000170153 | RNF150 | ring finger protein 150 [Source: HGNC |
| | | Symbol; Acc: HGNC: 23138] |
| ENSG00000155970 | MICU3 | mitochondrial calcium uptake family member 3 [Source: HGNC |
| | | Symbol; Acc: HGNC: 27820] |
| ENSG00000152495 | CAMK4 | calcium/calmodulin dependent protein kinase IV [Source: HGNC |
| | | Symbol; Acc: HGNC: 1464] |
| ENSG00000136404 | TM6SF1 | transmembrane 6 superfamily member 1 [Source: HGNC |
| | | Symbol; Acc: HGNC: 11860] |
| ENSG00000109339 | MAPK10 | mitogen-activated protein kinase 10 [Source: HGNC |
| | | Symbol; Acc: HGNC: 6872] |
| ENSG00000147100 | SLC16A2 | solute carrier family 16 member 2 [Source: HGNC |
| | | Symbol; Acc: HGNC: 10923] |
| ENSG00000162614 | NEXN | nexilin F-actin binding protein [Source: HGNC |
| | | Symbol; Acc: HGNC: 29557] |
| ENSG00000123096 | SSPN | sarcospan [Source: HGNC Symbol; Acc: HGNC: 11322] |
| ENSG00000184226 | PCDH9 | protocadherin 9 [Source: HGNC Symbol; Acc: HGNC: 8661] |
| ENSG00000174130 | TLR6 | toll like receptor 6 [Source: HGNC Symbol; Acc: HGNC: 16711] |
| ENSG00000189184 | PCDH18 | protocadherin 18 [Source: HGNC Symbol; Acc: HGNC: 14268] |
| ENSG00000048052 | HDAC9 | histone deacetylase 9 [Source: HGNC Symbol; Acc: HGNC: 14065] |
| ENSG00000154262 | ABCA6 | ATP binding cassette subfamily A member 6 [Source: HGNC |
| | | Symbol; Acc: HGNC: 36] |
| ENSG00000123094 | RASSF8 | Ras association domain family member 8 [Source: HGNC |
| | | Symbol; Acc: HGNC: 13232] |
| ENSG00000044524 | EPHA3 | EPH receptor A3 [Source: HGNC Symbol; Acc: HGNC: 3387] |
| ENSG00000198542 | ITGBL1 | integrin subunit beta like 1 [Source: HGNC |
| | | Symbol; Acc: HGNC: 6164] |
| ENSG00000120156 | TEK | TEK receptor tyrosine kinase [Source: HGNC |
| | | Symbol; Acc: HGNC: 11724] |
| ENSG00000064225 | ST3GAL6 | ST3 beta-galactoside alpha-2,3-sialyltransferase 6 [Source: HGNC |
| | | Symbol; Acc: HGNC: 18080] |
| ENSG00000152049 | KCNE4 | potassium voltage-gated channel subfamily E regulatory subunit 4 |
| | | [Source: HGNC Symbol; Acc: HGNC: 6244] |
| ENSG00000132357 | CARD6 | caspase recruitment domain family member 6 [Source: HGNC |
| | | Symbol; Acc: HGNC: 16394] |
| ENSG00000160593 | JAML | junction adhesion molecule like [Source: HGNC |
| | | Symbol; Acc: HGNC: 19084] |
| ENSG00000046889 | PREX2 | phosphatidylinositol-3,4,5-trisphosphate dependent Rac exchange |
| | | factor 2 [Source: HGNC Symbol; Acc: HGNC: 22950] |
| ENSG00000152527 | PLEKHH2 | pleckstrin homology, MyTH4 and FERM domain containing H2 |
| | | [Source: HGNC Symbol; Acc: HGNC: 30506] |
| ENSG00000111860 | CEP85L | centrosomal protein 85 like [Source: HGNC |
| | | Symbol; Acc: HGNC: 21638] |
| ENSG00000126785 | RHOJ | ras homolog family member J [Source: HGNC |
| | | Symbol; Acc: HGNC: 688] |
| ENSG00000134874 | DZIP1 | DAZ interacting zinc finger protein 1 [Source: HGNC |
| | | Symbol; Acc: HGNC: 20908] |
| ENSG00000168685 | IL7R | interleukin 7 receptor [Source: HGNC Symbol; Acc: HGNC: 6024] |
| ENSG00000111341 | MGP | matrix Gla protein [Source: HGNC Symbol; Acc: HGNC: 7060] |
| ENSG00000260314 | MRC1 | mannose receptor C-type 1 [Source: HGNC |
| | | Symbol; Acc: HGNC: 7228] |
| ENSG00000197872 | CYRIA | CYFIP related Rac1 interactor A [Source: HGNC |
| | | Symbol; Acc: HGNC: 25373] |
| ENSG00000105851 | PIK3CG | phosphatidylinositol-4,5-bisphosphate 3-kinase catalytic subunit |
| | | gamma [Source: HGNC Symbol; Acc: HGNC: 8978] |
| ENSG00000061918 | GUCY1B1 | guanylate cyclase 1 soluble subunit beta 1 [Source: HGNC |
| | | Symbol; Acc: HGNC: 4687] |
| ENSG00000078098 | FAP | fibroblast activation protein alpha [Source: HGNC |
| | | Symbol; Acc: HGNC: 3590] |
| ENSG00000186469 | GNG2 | G protein subunit gamma 2 [Source: HGNC |
| | | Symbol; Acc: HGNC: 4404] |
| ENSG00000187098 | MITF | melanocyte inducing transcription factor [Source: HGNC |
| | | Symbol; Acc: HGNC: 7105] |
| ENSG00000139926 | FRMD6 | FERM domain containing 6 [Source: HGNC |
| | | Symbol; Acc: HGNC: 19839] |
| ENSG00000104368 | PLAT | plasminogen activator, tissue type [Source: HGNC |
| | | Symbol; Acc: HGNC: 9051] |
| ENSG00000174099 | MSRB3 | methionine sulfoxide reductase B3 [Source: HGNC |
| | | Symbol; Acc: HGNC: 27375] |
| ENSG00000139329 | LUM | lumican [Source: HGNC Symbol; Acc: HGNC: 6724] |
| ENSG00000185340 | GAS2L1 | growth arrest specific 2 like 1 [Source: HGNC |
| | | Symbol; Acc: HGNC: 16955] |
| ENSG00000169744 | LDB2 | LIM domain binding 2 [Source: HGNC Symbol; Acc: HGNC: 6533] |
| ENSG00000104324 | CPQ | carboxypeptidase Q [Source: HGNC Symbol; Acc: HGNC: 16910] |
| ENSG00000139278 | GLIPR1 | GLI pathogenesis related 1 [Source: HGNC |
| | | Symbol; Acc: HGNC: 17001] |
| ENSG00000171488 | LRRC8C | leucine rich repeat containing 8 VRAC subunit C [Source: HGNC |
| | | Symbol; Acc: HGNC: 25075] |
| ENSG00000137393 | RNF144B | ring finger protein 144B [Source: HGNC |
| | | Symbol; Acc: HGNC: 21578] |
| ENSG00000213694 | S1PR3 | sphingosine-1-phosphate receptor 3 [Source: HGNC |
| | | Symbol; Acc: HGNC: 3167] |
| ENSG00000114859 | CLCN2 | chloride voltage-gated channel 2 [Source: HGNC |
| | | Symbol; Acc: HGNC: 2020] |
| ENSG00000140937 | CDH11 | cadherin 11 [Source: HGNC Symbol; Acc: HGNC: 1750] |
| ENSG00000082074 | FYB1 | FYN binding protein 1 [Source: HGNC Symbol; Acc: HGNC: 4036] |
| ENSG00000169439 | SDC2 | syndecan 2 [Source: HGNC Symbol; Acc: HGNC: 10659] |
| ENSG00000169604 | ANTXR1 | ANTXR cell adhesion molecule 1 [Source: HGNC |
| | | Symbol; Acc: HGNC: 21014] |
| ENSG00000081189 | MEF2C | myocyte enhancer factor 2C [Source: HGNC |
| | | Symbol; Acc: HGNC: 6996] |
| ENSG00000161618 | ALDH16A1 | aldehyde dehydrogenase 16 family member A1 [Source: HGNC |
| | | Symbol; Acc: HGNC: 28114] |
| ENSG00000178573 | MAF | MAF bZIP transcription factor [Source: HGNC |
| | | Symbol; Acc: HGNC: 6776] |
| ENSG00000111727 | HCFC2 | host cell factor C2 [Source: HGNC Symbol; Acc: HGNC: 24972] |
| ENSG00000099785 | MARCHF2 | membrane associated ring-CH-type finger 2 [Source: HGNC |
| | | Symbol; Acc: HGNC: 28038] |
| ENSG00000143341 | HMCN1 | hemicentin 1 [Source: HGNC Symbol; Acc: HGNC: 19194] |
| ENSG00000261221 | ZNF865 | zinc finger protein 865 [Source: HGNC |
| | | Symbol; Acc: HGNC: 38705] |
| ENSG00000158717 | RNF166 | ring finger protein 166 [Source: HGNC |
| | | Symbol; Acc: HGNC: 28856] |
| ENSG00000173264 | GPR137 | G protein-coupled receptor 137 [Source: HGNC |
| | | Symbol; Acc: HGNC: 24300] |
| ENSG00000175105 | ZNF654 | zinc finger protein 654 [Source: HGNC |
| | | Symbol; Acc: HGNC: 25612] |
| ENSG00000173482 | PTPRM | protein tyrosine phosphatase receptor type M [Source: HGNC |
| | | Symbol; Acc: HGNC: 9675] |
| ENSG00000184281 | TSSC4 | tumor suppressing subtransferable candidate 4 [Source: HGNC |
| | | Symbol; Acc: HGNC: 12386] |
| ENSG00000163453 | IGFBP7 | insulin like growth factor binding protein 7 [Source: HGNC |
| | | Symbol; Acc: HGNC: 5476] |
| ENSG00000112531 | QKI | QKI, KH domain containing RNA binding [Source: HGNC |
| | | Symbol; Acc: HGNC: 21100] |
| ENSG00000168876 | ANKRD49 | ankyrin repeat domain 49 [Source: HGNC |
| | | Symbol; Acc: HGNC: 25970] |
| ENSG00000100726 | TELO2 | telomere maintenance 2 [Source: HGNC |
| | | Symbol; Acc: HGNC: 29099] |
| ENSG00000119878 | CRIPT | CXXC repeat containing interactor of PDZ3 domain |
| | | [Source: HGNC Symbol; Acc: HGNC: 14312] |
| ENSG00000110719 | TCIRG1 | T cell immune regulator 1, ATPase H+ transporting V0 subunit a3 |
| | | [Source: HGNC Symbol; Acc: HGNC: 11647] |
| ENSG00000118762 | PKD2 | polycystin 2, transient receptor potential cation channel |
| | | [Source: HGNC Symbol; Acc: HGNC: 9009] |
| ENSG00000134954 | ETS1 | ETS proto-oncogene 1, transcription factor [Source: HGNC |
| | | Symbol; Acc: HGNC: 3488] |
| ENSG00000153130 | SCOC | short coiled-coil protein [Source: HGNC |
| | | Symbol; Acc: HGNC: 20335] |
| ENSG00000111711 | GOLT1B | golgi transport 1B [Source: HGNC Symbol; Acc: HGNC: 20175] |
| ENSG00000151665 | PIGF | phosphatidylinositol glycan anchor biosynthesis class F |
| | | [Source: HGNC Symbol; Acc: HGNC: 8962] |
| ENSG00000105321 | CCDC9 | coiled-coil domain containing 9 [Source: HGNC |
| | | Symbol; Acc: HGNC: 24560] |
| ENSG00000178177 | LCORL | ligand dependent nuclear receptor corepressor like [Source: HGNC |
| | | Symbol; Acc: HGNC: 30776] |
| ENSG00000014123 | UFL1 | UFM1 specific ligase 1 [Source: HGNC |
| | | Symbol; Acc: HGNC: 23039] |
| ENSG00000179387 | ELMOD2 | ELMO domain containing 2 [Source: HGNC |
| | | Symbol; Acc: HGNC: 28111] |
| ENSG00000126461 | SCAF1 | SR-related CTD associated factor 1 [Source: HGNC |
| | | Symbol; Acc: HGNC: 30403] |
| ENSG00000108406 | DHX40 | DEAH-box helicase 40 [Source: HGNC |
| | | Symbol; Acc: HGNC: 18018] |
| ENSG00000156017 | CARNMT1 | carnosine N-methyltransferase 1 [Source: HGNC |
| | | Symbol; Acc: HGNC: 23435] |
| ENSG00000120837 | NFYB | nuclear transcription factor Y subunit beta [Source: HGNC |
| | | Symbol; Acc: HGNC: 7805] |
| ENSG00000134352 | IL6ST | interleukin 6 signal transducer [Source: HGNC |
| | | Symbol; Acc: HGNC: 6021] |
| ENSG00000105722 | ERF | ETS2 repressor factor [Source: HGNC Symbol; Acc: HGNC: 3444] |
| ENSG00000168566 | SNRNP48 | small nuclear ribonucleoprotein U11/U12 subunit 48 |
| | | [Source: HGNC Symbol; Acc: HGNC: 21368] |
| ENSG00000095574 | IKZF5 | IKAROS family zinc finger 5 [Source: HGNC |
| | | Symbol; Acc: HGNC: 14283] |
| ENSG00000164323 | CFAP97 | cilia and flagella associated protein 97 [Source: HGNC |
| | | Symbol; Acc: HGNC: 29276] |
| ENSG00000180488 | MIGA1 | mitoguardin 1 [Source: HGNC Symbol; Acc: HGNC: 24741] |
| ENSG00000146282 | RARS2 | arginyl-tRNA synthetase 2, mitochondrial [Source: HGNC |
| | | Symbol; Acc: HGNC: 21406] |
| ENSG00000021574 | SPAST | spastin [Source: HGNC Symbol; Acc: HGNC: 11233] |
| ENSG00000164163 | ABCE1 | ATP binding cassette subfamily E member 1 [Source: HGNC |
| | | Symbol; Acc: HGNC: 69] |
| ENSG00000166200 | COPS2 | COP9 signalosome subunit 2 [Source: HGNC |
| | | Symbol; Acc: HGNC: 30747] |
| ENSG00000121879 | PIK3CA | phosphatidylinositol-4,5-bisphosphate 3-kinase catalytic subunit |
| | | alpha [Source: HGNC Symbol; Acc: HGNC: 8975] |
| ENSG00000149308 | NPAT | nuclear protein, coactivator of histone transcription |
| | | [Source: HGNC Symbol; Acc: HGNC: 7896] |
| ENSG00000146587 | RBAK | RB associated KRAB zinc finger [Source: HGNC |
| | | Symbol; Acc: HGNC: 17680] |
| ENSG00000141101 | NOB1 | NIN1 (RPN12) binding protein 1 homolog [Source: HGNC |
| | | Symbol; Acc: HGNC: 29540] |
| ENSG00000135974 | C2orf49 | chromosome 2 open reading frame 49 [Source: HGNC |
| | | Symbol; Acc: HGNC: 28772] |
| ENSG00000138138 | ATAD1 | ATPase family AAA domain containing 1 [Source: HGNC |
| | | Symbol; Acc: HGNC: 25903] |
| ENSG00000115827 | DCAF17 | DDB1 and CUL4 associated factor 17 [Source: HGNC |
| | | Symbol; Acc: HGNC: 25784] |
| ENSG00000125503 | PPP1R12C | protein phosphatase 1 regulatory subunit 12C [Source: HGNC |
| | | Symbol; Acc: HGNC: 14947] |
| ENSG00000129317 | PUS7L | pseudouridine synthase 7 like [Source: HGNC |
| | | Symbol; Acc: HGNC: 25276] |
| ENSG00000126391 | FRMD8 | FERM domain containing 8 [Source: HGNC |
| | | Symbol; Acc: HGNC: 25462] |
| ENSG00000115816 | CEBPZ | CCAAT enhancer binding protein zeta [Source: HGNC |
| | | Symbol; Acc: HGNC: 24218] |
| ENSG00000149499 | EML3 | EMAP like 3 [Source: HGNC Symbol; Acc: HGNC: 26666] |
| ENSG00000164327 | RICTOR | RPTOR independent companion of MTOR complex 2 |
| | | [Source: HGNC Symbol; Acc: HGNC: 28611] |
| ENSG00000108819 | PPP1R9B | protein phosphatase 1 regulatory subunit 9B [Source: HGNC |
| | | Symbol; Acc: HGNC: 9298] |
| ENSG00000119414 | PPP6C | protein phosphatase 6 catalytic subunit [Source: HGNC |
| | | Symbol; Acc: HGNC: 9323] |
| ENSG00000132510 | KDM6B | lysine demethylase 6B [Source: HGNC |
| | | Symbol; Acc: HGNC: 29012] |
| ENSG00000148943 | LIN7C | lin-7 homolog C, crumbs cell polarity complex component |
| | | [Source: HGNC Symbol; Acc: HGNC: 17789] |
| ENSG00000167005 | NUDT21 | nudix hydrolase 21 [Source: HGNC Symbol; Acc: HGNC: 13870] |
| ENSG00000162664 | ZNF326 | zinc finger protein 326 [Source: HGNC |
| | | Symbol; Acc: HGNC: 14104] |
| ENSG00000122545 | SEPTIN7 | septin 7 [Source: HGNC Symbol; Acc: HGNC: 1717] |
| ENSG00000138078 | PREPL | prolyl endopeptidase like [Source: HGNC |
| | | Symbol; Acc: HGNC: 30228] |
| ENSG00000168813 | ZNF507 | zinc finger protein 507 [Source: HGNC |
| | | Symbol; Acc: HGNC: 23783] |
| ENSG00000104805 | NUCB1 | nucleobindin 1 [Source: HGNC Symbol; Acc: HGNC: 8043] |
| ENSG00000114416 | FXR1 | FMR1 autosomal homolog 1 [Source: HGNC |
| | | Symbol; Acc: HGNC: 4023] |
| ENSG00000136536 | MARCHF7 | membrane associated ring-CH-type finger 7 [Source: HGNC |
| | | Symbol; Acc: HGNC: 17393] |
| ENSG00000163714 | U2SURP | U2 snRNP associated SURP domain containing [Source: HGNC |
| | | Symbol; Acc: HGNC: 30855] |
| ENSG00000096746 | HNRNPH3 | heterogeneous nuclear ribonucleoprotein H3 [Source: HGNC |
| | | Symbol; Acc: HGNC: 5043] |
| ENSG00000105397 | TYK2 | tyrosine kinase 2 [Source: HGNC Symbol; Acc: HGNC: 12440] |
| ENSG00000118260 | CREB1 | cAMP responsive element binding protein 1 [Source: HGNC |
| | | Symbol; Acc: HGNC: 2345] |
| ENSG00000146247 | PHIP | pleckstrin homology domain interacting protein [Source: HGNC |
| | | Symbol; Acc: HGNC: 15673] |
| ENSG00000135486 | HNRNPA1 | heterogeneous nuclear ribonucleoprotein A1 [Source: HGNC |
| | | Symbol; Acc: HGNC: 5031] |
| ENSG00000163785 | RYK | receptor like tyrosine kinase [Source: HGNC |
| | | Symbol; Acc: HGNC: 10481] |
| ENSG00000198586 | TLK1 | tousled like kinase 1 [Source: HGNC Symbol; Acc: HGNC: 11841] |
| ENSG00000118007 | STAG1 | stromal antigen 1 [Source: HGNC Symbol; Acc: HGNC: 11354] |
| ENSG00000138081 | FBXO11 | F-box protein 11 [Source: HGNC Symbol; Acc: HGNC: 13590] |
| ENSG00000090060 | PAPOLA | poly(A) polymerase alpha [Source: HGNC |
| | | Symbol; Acc: HGNC: 14981] |
| ENSG00000244462 | RBM12 | RNA binding motif protein 12 [Source: HGNC |
| | | Symbol; Acc: HGNC: 9898] |
| ENSG00000162613 | FUBP1 | far upstream element binding protein 1 [Source: HGNC |
| | | Symbol; Acc: HGNC: 4004] |
| ENSG00000085224 | ATRX | ATRX chromatin remodeler [Source: HGNC |
| | | Symbol; Acc: HGNC: 886] |
| ENSG00000011405 | PIK3C2A | phosphatidylinositol-4-phosphate 3-kinase catalytic subunit |
| | | type 2 alpha [Source: HGNC Symbol; Acc: HGNC: 8971] |
| ENSG00000048649 | RSF1 | remodeling and spacing factor 1 [Source: HGNC |
| | | Symbol; Acc: HGNC: 18118] |
| ENSG00000112739 | PRPF4B | pre-mRNA processing factor 4B [Source: HGNC |
| | | Symbol; Acc: HGNC: 17346] |
| ENSG00000133704 | IPO8 | importin 8 [Source: HGNC Symbol; Acc: HGNC: 9853] |
| ENSG00000112701 | SENP6 | SUMO specific peptidase 6 [Source: HGNC |
| | | Symbol; Acc: HGNC: 20944] |
| ENSG00000129315 | CCNT1 | cyclin T1 [Source: HGNC Symbol; Acc: HGNC: 1599] |
| ENSG00000168958 | MFF | mitochondrial fission factor [Source: HGNC |
| | | Symbol; Acc: HGNC: 24858] |
| ENSG00000075292 | ZNF638 | zinc finger protein 638 [Source: HGNC |
| | | Symbol; Acc: HGNC: 17894] |
| ENSG00000156976 | EIF4A2 | eukaryotic translation initiation factor 4A2 [Source: HGNC |
| | | Symbol; Acc: HGNC: 3284] |
| ENSG00000164190 | NIPBL | NIPBL cohesin loading factor [Source: HGNC |
| | | Symbol; Acc: HGNC: 28862] |
| ENSG00000115464 | USP34 | ubiquitin specific peptidase 34 [Source: HGNC |
| | | Symbol; Acc: HGNC: 20066] |
| ENSG00000145495 | MARCHF6 | membrane associated ring-CH-type finger 6 [Source: HGNC |
| | | Symbol; Acc: HGNC: 30550] |
| ENSG00000106263 | EIF3B | eukaryotic translation initiation factor 3 subunit B [Source: |
| | | HGNC Symbol; Acc: HGNC: 3280] |
| ENSG00000114978 | MOB1A | MOB kinase activator 1A [Source: HGNC |
| | | Symbol; Acc: HGNC: 16015] |
| ENSG00000114933 | INO80D | INO80 complex subunit D [Source: HGNC |
| | | Symbol; Acc: HGNC: 25997] |
| ENSG00000147274 | RBMX | RNA binding motif protein X-linked [Source: HGNC |
| | | Symbol; Acc: HGNC: 9910] |
| ENSG00000135870 | RC3H1 | ring finger and CCCH-type domains 1 [Source: HGNC |
| | | Symbol; Acc: HGNC: 29434] |
| ENSG00000122566 | HNRNPA2B1 | heterogeneous nuclear ribonucleoprotein A2/B1 [Source: HGNC |
| | | Symbol; Acc: HGNC: 5033] |

### Example 2—Neural Network Classifier

The inventors developed a neural network classifier based on expression of the 150 genes identified in Table 1. In summary, the expression feature inputs (X) from a sample plus a column of 1's get matrix multiplied by a transposed Theta1 (see Table 2 below), and this gives the matrix h1. This matrix is then fed into a sigmoid function and the output plus a column of 1's gets multiplied by the transposed Theta2 (see Table 2 below) and fed to a sigmoid. The final result is a column vector of three probabilities giving the probability of subtype 1 (canonical), 2 (immune), or 3 (stromal). The final subtype classification output is determined by assigning the sample to the class corresponding to the highest probability.

The Theta1 matrices have an additional column that corresponds to the bias term. This is a constant feature input that is always 1, it is analogous to a constant term for a linear or logistic regression. The Theta 2 matrices also have 36 columns corresponding to the 35 neurons used in the hidden layer plus an additional bias term of 1. The inputs to the output layer is the output of the hidden layer plus the constant bias term. That input is fed into 3 output neurons that give the probability of the sample being of class 1 (canonical), 2 (immune), or 3 (stromal). Below is the matlab code used to calculate the prediction:
function p=predict(Theta1, Theta2, X)
%PREDICT Predict the label of an input given a trained neural network
% p =PREDICT(Theta1, Theta2, X) outputs the predicted label of X given the
% trained weights of a neural network (Theta1, Theta2)
% Useful values
m=size(X, 1);
num_labels=size(Theta2, 1);
% You need to return the following variables correctly
p=zeros(size(X, 1), 1);
h1=sigmoid([ones(m, 1) X]*Theta1′);
h2=sigmoid([ones(m, 1) h1]*Theta2′);
[dummy, p]=max(h2, [], 2);
%
   end

TABLE 2
Theta1 and Theta2 for Neural Network Classifier

| Column | | | Column | |
|---|---|---|---|---|
| ID | | Gene | ID | |
| (Theta 1) | Feature | Symbol | (Theta 2) | Feature |
| A | Bias term | | A | Bias term |
| B | ENSG00000204381 | LAYN | B | Node 1 |
| C | ENSG00000170153 | RNF150 | C | Node 2 |
| D | ENSG00000155970 | MICU3 | D | Node 3 |
| E | ENSG00000152495 | CAMK4 | E | Node 4 |
| F | ENSG00000136404 | TM6SF1 | F | Node 5 |
| G | ENSG00000109339 | MAPK10 | G | Node 6 |
| H | ENSG00000147100 | SLC16A2 | H | Node 7 |
| I | ENSG00000162614 | NEXN | I | Node 8 |
| J | ENSG00000123096 | SSPN | J | Node 9 |
| K | ENSG00000184226 | PCDH9 | K | Node 10 |
| L | ENSG00000174130 | TLR6 | L | Node 11 |
| M | ENSG00000189184 | PCDH18 | M | Node 12 |
| N | ENSG00000048052 | HDAC9 | N | Node 13 |
| O | ENSG00000154262 | ABCA6 | O | Node 14 |
| P | ENSG00000123094 | RASSF8 | P | Node 15 |
| Q | ENSG00000044524 | EPHA3 | Q | Node 16 |
| R | ENSG00000198542 | ITGBL1 | R | Node 17 |
| S | ENSG00000120156 | TEK | S | Node 18 |
| T | ENSG00000064225 | ST3GAL6 | T | Node 19 |
| U | ENSG00000152049 | KCNE4 | U | Node 20 |
| V | ENSG00000132357 | CARD6 | V | Node 21 |
| W | ENSG00000160593 | JAML | W | Node 22 |
| X | ENSG00000046889 | PREX2 | X | Node 23 |
| Y | ENSG00000152527 | PLEKHH2 | Y | Node 24 |
| Z | ENSG00000111860 | CEP85L | Z | Node 25 |
| AA | ENSG00000126785 | RHOJ | AA | Node 26 |
| AB | ENSG00000134874 | DZIP1 | AB | Node 27 |
| AC | ENSG00000168685 | IL7R | AC | Node 28 |
| AD | ENSG00000111341 | MGP | AD | Node 29 |
| AE | ENSG00000260314 | MRC1 | AE | Node 30 |
| AF | ENSG00000197872 | CYRIA | AF | Node 31 |
| AG | ENSG00000105851 | PIK3CG | AG | Node 32 |
| AH | ENSG00000061918 | GUCY1B1 | AH | Node 33 |
| AI | ENSG00000078098 | FAP | AI | Node 34 |
| AJ | ENSG00000186469 | GNG2 | AJ | Node 35 |
| AK | ENSG00000187098 | MITF | — | — |
| AL | ENSG00000139926 | FRMD6 | — | — |
| AM | ENSG00000104368 | PLAT | — | — |
| AN | ENSG00000174099 | MSRB3 | — | — |
| AO | ENSG00000139329 | LUM | — | — |
| AP | ENSG00000185340 | GAS2L1 | — | — |
| AQ | ENSG00000169744 | LDB2 | — | — |
| AR | ENSG00000104324 | CPQ | — | — |
| AS | ENSG00000139278 | GLIPR1 | — | — |
| AT | ENSG00000171488 | LRRC8C | — | — |
| AU | ENSG00000137393 | RNF144B | — | — |
| AV | ENSG00000213694 | S1PR3 | — | — |
| AW | ENSG00000114859 | CLCN2 | — | — |
| AX | ENSG00000140937 | CDH11 | — | — |
| AY | ENSG00000082074 | FYB1 | — | — |
| AZ | ENSG00000169439 | SDC2 | — | — |
| BA | ENSG00000169604 | ANTXR1 | — | — |
| BB | ENSG00000081189 | MEF2C | — | — |
| BC | ENSG00000161618 | ALDH16A1 | — | — |
| BD | ENSG00000178573 | MAF | — | — |
| BE | ENSG00000111727 | HCFC2 | — | — |
| BF | ENSG00000099785 | MARCHF2 | — | — |
| BG | ENSG00000143341 | HMCN1 | — | — |
| BH | ENSG00000261221 | ZNF865 | — | — |
| BI | ENSG00000158717 | RNF166 | — | — |
| BJ | ENSG00000173264 | GPR137 | — | — |
| BK | ENSG00000175105 | ZNF654 | — | — |
| BL | ENSG00000173482 | PTPRM | — | — |
| BM | ENSG00000184281 | TSSC4 | — | — |
| BN | ENSG00000163453 | IGFBP7 | — | — |
| BO | ENSG00000112531 | QKI | — | — |
| BP | ENSG00000168876 | ANKRD49 | — | — |
| BQ | ENSG00000100726 | TELO2 | — | — |
| BR | ENSG00000119878 | CRIPT | — | — |
| BS | ENSG00000110719 | TCIRG1 | — | — |
| BT | ENSG00000118762 | PKD2 | — | — |
| BU | ENSG00000134954 | ETS1 | — | — |
| BV | ENSG00000153130 | SCOC | — | — |
| BW | ENSG00000111711 | GOLT1B | — | — |
| BX | ENSG00000151665 | PIGF | — | — |
| BY | ENSG00000105321 | CCDC9 | — | — |
| BZ | ENSG00000178177 | LCORL | — | — |
| CA | ENSG00000014123 | UFL1 | — | — |
| CB | ENSG00000179387 | ELMOD2 | — | — |
| CC | ENSG00000126461 | SCAF1 | — | — |
| CD | ENSG00000108406 | DHX40 | — | — |
| CE | ENSG00000156017 | CARNMT1 | — | — |
| CF | ENSG00000120837 | NFYB | — | — |
| CG | ENSG00000134352 | IL6ST | — | — |
| CH | ENSG00000105722 | ERF | — | — |
| CI | ENSG00000168566 | SNRNP48 | — | — |
| CJ | ENSG00000095574 | IKZF5 | — | — |
| CK | ENSG00000164323 | CFAP97 | — | — |
| CL | ENSG00000180488 | MIGA1 | — | — |
| CM | ENSG00000146282 | RARS2 | — | — |
| CN | ENSG00000021574 | SPAST | — | — |
| CO | ENSG00000164163 | ABCE1 | — | — |
| CP | ENSG00000166200 | COPS2 | — | — |
| CQ | ENSG00000121879 | PIK3CA | — | — |
| CR | ENSG00000149308 | NPAT | — | — |
| CS | ENSG00000146587 | RBAK | — | — |
| CT | ENSG00000141101 | NOB1 | — | — |
| CU | ENSG00000135974 | C2orf49 | — | — |
| CV | ENSG00000138138 | ATAD1 | — | — |
| CW | ENSG00000115827 | DCAF17 | — | — |
| CX | ENSG00000125503 | PPP1R12C | — | — |
| CY | ENSG00000129317 | PUS7L | — | — |
| CZ | ENSG00000126391 | FRMD8 | — | — |
| DA | ENSG00000115816 | CEBPZ | — | — |
| DB | ENSG00000149499 | EML3 | — | — |
| DC | ENSG00000164327 | RICTOR | — | — |
| DD | ENSG00000108819 | PPP1R9B | — | — |
| DE | ENSG00000119414 | PPP6C | — | — |
| DF | ENSG00000132510 | KDM6B | — | — |
| DG | ENSG00000148943 | LIN7C | — | — |
| DH | ENSG00000167005 | NUDT21 | — | — |
| DI | ENSG00000162664 | ZNF326 | — | — |
| DJ | ENSG00000122545 | SEPTIN7 | — | — |
| DK | ENSG00000138078 | PREPL | — | — |
| DL | ENSG00000168813 | ZNF507 | — | — |
| DM | ENSG00000104805 | NUCB1 | — | — |
| DN | ENSG00000114416 | FXR1 | — | — |
| DO | ENSG00000136536 | MARCHF7 | — | — |
| DP | ENSG00000163714 | U2SURP | — | — |
| DQ | ENSG00000096746 | HNRNPH3 | — | — |
| DR | ENSG00000105397 | TYK2 | — | — |
| DS | ENSG00000118260 | CREB1 | — | — |
| DT | ENSG00000146247 | PHIP | — | — |
| DU | ENSG00000135486 | HNRNPA1 | — | — |
| DV | ENSG00000163785 | RYK | — | — |
| DW | ENSG00000198586 | TLK1 | — | — |
| DX | ENSG00000118007 | STAG1 | — | — |
| DY | ENSG00000138081 | FBXO11 | — | — |
| DZ | ENSG00000090060 | PAPOLA | — | — |
| CA | ENSG00000244462 | RBM12 | — | — |
| CB | ENSG00000162613 | FUBP1 | — | — |
| CC | ENSG00000085224 | ATRX | — | — |
| CD | ENSG00000011405 | PIK3C2A | — | — |
| CE | ENSG00000048649 | RSF1 | — | — |
| CF | ENSG00000112739 | PRPF4B | — | — |
| CG | ENSG00000133704 | IPO8 | — | — |
| CH | ENSG00000112701 | SENP6 | — | — |
| CI | ENSG00000129315 | CCNT1 | — | — |
| CJ | ENSG00000168958 | MFF | — | — |
| CK | ENSG00000075292 | ZNF638 | — | — |
| CL | ENSG00000156976 | EIF4A2 | — | — |
| CM | ENSG00000164190 | NIPBL | — | — |
| CN | ENSG00000115464 | USP34 | — | — |
| CO | ENSG00000145495 | MARCHF6 | — | — |
| CP | ENSG00000106263 | EIF3B | — | — |
| CQ | ENSG00000114978 | MOB1A | — | — |
| CR | ENSG00000114933 | INO80D | — | — |
| CS | ENSG00000147274 | RBMX | — | — |
| CT | ENSG00000135870 | RC3H1 | — | — |

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Hellman S, Weichselbaum R R. Oligometastases. J Clin Oncol 1995; 13:8-10.
2. Weichselbaum R R, Hellman S. Oligometastases revisited. Nat Rev Clin Oncol 2011; 8:378-82.
3. Palma D A, Salama J K, Lo S S, et al. The oligometastatic state—separating truth from wishful thinking. Nat Rev Clin Oncol 2014; 11:549-57.
4. Treasure T. Oligometastatic cancer: an entity, a useful concept, or a therapeutic opportunity? J R Soc Med 2012; 105:242-6.
5. Mehta N, Mauer A M, Hellman S, et al. Analysis of further disease progression in metastatic non-small cell lung cancer: implications for locoregional treatment. Int J Oncol 2004; 25:1677-83.
6. Hong J C, Salama J K. The expanding role of stereotactic body radiation therapy in oligometastatic solid tumors: What do we know and where are we going? Cancer Treat Rev 2017; 52:22-32.
7. Tosoian J J, Gorin M A, Ross A E, Pienta K J, Tran P T, Schaeffer E M. Oligometastatic prostate cancer: definitions, clinical outcomes, and treatment considerations. Nat Rev Urol 2017; 14:15-25.
8. Loh J, Davis I D, Martin J M, Siva S. Extracranial oligometastatic renal cell carcinoma: current management and future directions. Future Oncol 2014; 10:761-74.
9. Fong Y, Fortner J, Sun R L, Brennan M F, Blumgart L H. Clinical score for predicting recurrence after hepatic resection for metastatic colorectal cancer: analysis of 1001 consecutive cases. Ann Surg 1999; 230:309-18; discussion 18-21.
10. Tomlinson J S, Jarnagin W R, DeMatteo R P, et al. Actual 10-year survival after resection of colorectal liver metastases defines cure. J Clin Oncol 2007; 25:4575-80.
11. Kadri S, Long B C, Mujacic I, et al. Clinical Validation of a Next-Generation Sequencing Genomic Oncology Panel via Cross-Platform Benchmarking against Established Amplicon Sequencing Assays. J Mol Diagn 2017; 19:43-56.
12. Mann C D, Metcalfe M S, Leopardi L N, Maddern G J. The clinical risk score: emerging as a reliable preoperative prognostic index in hepatectomy for colorectal metastases. Arch Surg 2004; 139:1168-72.
13. Ivanecz A, Potrc S, Horvat M, Jagric T, Gadzijev E. The validity of clinical risk score for patients undergoing liver resection for colorectal metastases. Hepatogastroenterology 2009; 56:1452-8.
14. Pitroda et al., “Integrated molecular subtyping defines a curable oligometastatic state in colorectal liver metastasis,” Nature Communications 9:1793 (2018).
15. PCT Publication WO2019/204576 to Pitroda et al.
16. Duda et al., 2001, Pattern Classification, Second Edition, John Wiley & Sons, Inc., New York.
17. Hastie et al., 2001, The Elements of Statistical Learning, Springer-Verlag, New York.

What is claimed is:

1. A method of analyzing a tissue sample comprising measuring expression levels of one or more genes listed in Table 1 in a sample comprising tissue from a metastasis from a primary cancer tumor.

2. The method of claim 1, wherein the expression levels of at least two of the genes listed in Table 1 are measured.

3. The method of claim 1, wherein the expression levels of at least five of the genes listed in Table 1 are measured.

4. The method of claim 1, wherein the expression levels of at least ten of the genes listed in Table 1 are measured.

5. The method of claim 1, wherein the expression levels of at least twenty of the genes listed in Table 1 are measured.

6. The method of claim 1, wherein the expression levels of at least fifty of the genes listed in Table 1 are measured.

7. The method of claim 1, wherein the expression levels of all of the genes listed in Table 1 are measured.

8. The method of claim 1, wherein no expression levels of genes are measured other than those listed in Table 1.

9. The method of any of claims 1-8, wherein the metastasis is a liver metastasis.

10. The method of any of claims 1-8, wherein the primary cancer tumor is a colorectal cancer tumor.

11. The method of any of claims 1-10, wherein the expression levels of the one or more genes are within a predetermined amount of a mean expression level in metastases of a cohort of patients having one of the following three metastatic phenotypes: canonical, immune, or stromal.

12. The method of any of claims 1-11, further comprising calculating a clinical risk score for the patient.

13. The method of any of claims 1-12, further comprising analyzing the expression levels of the one or more genes using a multi-layer neural network classification process that includes an input layer, one or more hidden layers, and an output layer.

14. The method of claim 13, wherein the input layer comprises the expression levels of the one or more genes.

15. The method of claim 13 or 14, wherein the output layer comprises a classification of the expression level data of the input layer as indicating a canonical, an immune, or a stromal metastatic phenotype.

16. The method of any of claims 13-15, wherein the classification process comprises determining the probability that the metastasis has a canonical, immune, or stromal metastatic phenotype.

17. The method of claim 16, wherein the classification process comprises determining each of the three probabilities of the metastasis having a canonical, immune, and metastatic phenotype.

18. The method of any of claims 13-17, wherein the neural network classification process comprises a first hidden layer and a second hidden layer.

19. The method of any of claims 1-18, further comprising, prior to measuring the expression levels, obtaining the sample from a subject.

20. The method of any of claims 1-18, wherein the sample is from a subject.

21. The method of claim 20 or 21, further comprising administering a cancer therapy to the subject.

22. The method of claim 21, wherein the cancer therapy comprises a local cancer therapy and does not comprise a systemic cancer therapy.

23. The method of claim 21, wherein the cancer therapy comprises an immunotherapy.

24. The method of any of claims 1-23, wherein measuring the expression levels of the one or more genes comprises RNA sequencing.

25. The method of any of claims 1-23, wherein measuring the expression levels of the one or more genes comprises a microarray.

26. The method of any of claims 1-23, wherein measuring the expression levels of the one or more genes comprises performing polymerase chain reaction.

27. A method of analyzing a tissue sample comprising measuring expression levels of all of the genes of Table 1 in a sample comprising tissue from a metastasis from a primary cancer tumor.

28. A method of analyzing a tissue sample comprising measuring expression levels of all of LAYN, RNF150, MICU3, CAMK4, TM6SF1, MAPK10, SLC16A2, NEXN, SSPN, PCDH9, TLR6, PCDH18, HDAC9, ABCA6, RASSF8, EPHA3, ITGBL1, TEK, ST3GAL6, KCNE4, CARD6, JAML, PREX2, PLEKHH2, CEP85L, RHOJ, DZIP1, IL7R, MGP, MRC1, CYRIA, PIK3CG, GUCY1B1, FAP, GNG2, MITF, FRMD6, PLAT, MSRB3, LUM, GAS2L1, LDB2, CPQ, GLIPR1, LRRC8C, RNF144B, S1PR3, CLCN2, CDH11, FYB1, SDC2, ANTXR1, MEF2C, ALDH16A1, MAF, HCFC2, MARCHF2, HMCN1, ZNF865, RNF166, GPR137, ZNF654, PTPRM, TSSC4, IGFBP7, QKI, ANKRD49, TELO2, CRIPT, TCIRG1, PKD2, ETS1, SCOC, GOLT1B, PIGF, CCDC9, LCORL, UFL1, ELMOD2, SCAF1, DHX40, CARNMT1, NFYB, IL6ST, ERF, SNRNP48, IKZF5, CFAP97, MIGA1, RARS2, SPAST, ABCE1, COPS2, PIK3CA, NPAT, RBAK, NOB1, C2orf49, ATAD1, DCAF17, PPP1R12C, PUS7L, FRMD8, CEBPZ, EML3, RICTOR, PPP1R9B, PPP6C, KDM6B, LIN7C, NUDT21, ZNF326, SEPTIN7, PREPL, ZNF507, NUCB1, FXR1, MARCHF7, U2SURP, HNRNPH3, TYK2, CREB1, PHIP, HNRNPA1, RYK, TLK1, STAG1, FBXO11, PAPOLA, RBM12, FUBP1, ATRX, PIK3C2A, RSF1, PRPF4B, IP08, SENP6, CCNT1, MFF, ZNF638, EIF4A2, NIPBL, USP34, MARCHF6, EIF3B, MOB1A, INO80D, RBMX, RC3H1, and HNRNPA2B1 in a sample comprising tissue from a metastasis from a primary cancer tumor.

29. A method of treating metastatic cancer in a patient, the method comprising administering to the patient a local cancer therapy without administering systemic cancer therapy, administering to the patient an immunotherapy, or administering to the patient an EGFR inhibitor, wherein the patient has been determined to have a metastasis having expression levels of one or more genes listed in Table 1 that indicate a canonical or immune metastatic phenotype based on a multi-layer neural network classification process.

30. The method of claim 29, wherein the multi-layer neural network classification process comprises an input layer, one or more hidden layers, and an output layer.

31. The method of claim 30, wherein the input layer comprises the expression levels of the one or more genes.

32. The method of claim 31, wherein the input layer comprises the expression levels of at least two of the genes listed in Table 1.

33. The method of claim 31, wherein the input layer comprises the expression levels of all of the genes listed in Table 1.

34. The method of any of claims 30-33, wherein the output layer comprises a classification of the expression level data of the input layer as indicating a canonical, an immune, or a stromal metastatic phenotype.

35. A method of treating metastatic cancer in a patient, the method comprising administering to the patient a local cancer therapy without administering systemic cancer therapy or administering to the patient an immunotherapy or EGFR inhibitor, wherein the patient has been determined to have a metastasis having expression levels of one or more genes listed in Table 1 that are within a predetermined amount of the mean expression level of the one or more genes in metastases of a cohort of metastatic cancer patients having a mean overall five-year survival expectation that is at least 60% or a mean five-year disease-free survival expectation that is at least 30%.

36. The method of claim 35, wherein the patient has been determined to have a metastasis having expression levels of at least two of the genes listed in Table 1 that are within predetermined amounts of the mean expression levels of the genes in metastases of a cohort of metastatic cancer patients having a mean overall five-year survival expectation that is at least 60% or a mean five-year disease-free survival expectation that is at least 30%.

37. The method of claim 35, wherein the patient has been determined to have a metastasis having expression levels of all of the genes listed in Table 1 that are within predetermined amounts of the mean expression levels of the genes in metastases of a cohort of metastatic cancer patients having a mean overall five-year survival expectation that is at least 60% or a mean five-year disease-free survival expectation that is at least 30%.

38. The method of claim 37, wherein the expression levels of the one or more genes indicate a canonical or immune metastatic phenotype.

39. The method of claim 37 or 38, wherein an expression signature of the one or more genes matches an expression signature of a canonical or immune metastatic phenotype.

40. The method of any one of claims 37 to 39, wherein the expression levels of the one or more genes have been used as an input layer of a multi-layer neural network classification system.

41. A method of treating cancer in a patient having a metastasis from a primary cancer tumor, the method comprising: administering to the patient an immune checkpoint therapy or administering to the patient a local cancer therapy without administering a systemic cancer therapy, wherein the patient has been identified based on expression levels of one or more genes in the metastasis as belonging to a group of metastatic cancer patients with one or more of the following characteristics:
(a) a mean five-year overall survival expectation of at least 60%;
(b) a mean five-year disease-free survival expectation of at least 30%;
(c) a likelihood of experiencing metastatic recurrence after hepatic resection that is lower than the likelihood for patients outside of the group;
(d) a canonical metastatic phenotype; and
(e) an immune metastatic phenotype.

42. The method of claim 41, wherein the one or more genes comprise two or more of the genes listed in Table 1.

43. The method of claim 41, wherein the one or more genes comprise five or more of the genes listed in Table 1.

44. The method of claim 41, wherein the one or more genes comprise ten or more of the genes listed in Table 1.

45. The method of claim 41, wherein the one or more genes comprise twenty or more of the genes listed in Table 1.

46. The method of claim 41, wherein the one or more genes comprise fifty or more of the genes listed in Table 1.

47. The method of claim 41, wherein the one or more genes comprise all of the genes listed in Table 1.

48. The method of claim 41, wherein the one or more genes do not comprise transcripts of any genes other than those listed in Table 1.

49. The method of any of claims 41-48, wherein the metastasis is a liver metastasis and the cancer is colorectal cancer.

50. A method of diagnosing a patient having a metastasis from a primary colorectal cancer tumor, the method comprising:
(a) determining expression levels in the metastasis of one or more of the genes listed in Table 1;
(b) identifying the patient as having a canonical metastatic phenotype, as having an immune metastatic phenotype, as being a responder to immune checkpoint cancer therapy, as having a five-year overall survival expectation of greater than 60%, or as having a five-year disease-free survival expectation of greater than 30% if the expression level of one or more of the genes is within a predetermined amount of a first reference expression level or deviates from a second reference expression level by a predetermined amount.

51. The method of claim 50, wherein the first reference expression level represents the mean expression level in metastases of a cohort of metastatic cancer patients having a canonical metastatic phenotype, having an immune metastatic phenotype, being a responders to immune checkpoint cancer therapy, having a five-year overall survival expectation of greater than 60%, and/or having a five-year disease-free survival expectation of greater than 30%.

52. The method of claim 50 or 51, wherein the second reference expression level represents the mean expression level in metastases of a cohort of metastatic cancer patients having a mean five-year overall survival expectation of less than 60%.

53. The method of any of claims 50-52, wherein (a) comprises determining expression levels in the metastasis of all of the genes listed in Table 1.

54. A method of treating a patient having a metastasis from a primary colorectal cancer tumor, the method comprising:
(a) measuring the expression of one or more genes in a sample from the metastasis;
(b) comparing the measured expression level of each gene to a reference expression level for that gene;
(c) identifying the metastasis as having a canonical, immune, or stromal phenotype based on the measured expression levels; and
(d) administering to the patient an appropriate therapy based on the type of metastasis identified in step (c).

55. The method of claim 54, wherein (a) comprises measuring the expression of at least two of the genes listed in Table 1.

56. The method of claim 54, wherein (a) comprises measuring the expression of at least five of the genes listed in Table 1.

57. The method of claim 54, wherein (a) comprises measuring the expression of at least ten of the genes listed in Table 1.

58. The method of claim 54, wherein (a) comprises measuring the expression of at least twenty of the genes listed in Table 1.

59. The method of claim 54, wherein (a) comprises measuring the expression of at least fifty of the genes listed in Table 1.

60. The method of claim 54, wherein (a) comprises measuring the expression of all of the genes listed in Table 1.

61. The method of any of claims 54-60, wherein (b) comprises analyzing the expression level of each gene using a multi-layer neural network classification system having an input layer, one or more hidden layers, and an output layer, wherein the input layer comprises the expression levels of the one or more genes and wherein the output layer comprises a classification of the expression level data of the input layer as indicating a canonical, an immune, or a stromal metastatic phenotype.

62. The method of any of claims 54-61, wherein the appropriate therapy for a patient with a canonical-type metastasis comprises a DNA damaging chemotherapy, PARP inhibitor, angiogenesis inhibitor, or MYC inhibitor.

63. The method of any of claims 54-61, wherein the appropriate therapy for a patient with an immune-type metastasis comprises an EGFR inhibitor, immunotherapy, or a splicing inhibitor.

64. The method of any of claims 54-61, wherein the appropriate therapy for a patient with a stromal-type metastasis comprises an angiogenesis inhibitor, KRAS inhibitor, or tumor stromal inhibitor, or excludes an EGFR inhibitor.

65. A method of treating a patient having metastatic colorectal cancer, the method comprising administering an EGFR inhibitor to a patient who has been tested and found to have liver metastases of an immune molecular subtype by analyzing the expression levels of transcripts of at least two of the genes listed in Table 1.

66. The method of claim 65, wherein the expression levels of the genes are analyzed using a neural network classification process.

67. The method of claim 65 or 66, wherein the input into the neural network classification process consists of all the genes listed in Table 1.

68. The method of any of claims 65-67, wherein the input into the neural network classification process includes only genes listed in Table 1.

69. The method of any of claims 65-68, wherein the EGFR inhibitor is cetuximab.

70. A method of diagnosing a patient having a liver metastasis from a primary colorectal cancer tumor, the method comprising inputting the expression levels in the metastasis of one or more of the genes listed on Table 1 into a classifier that has been trained to recognize an expression signature of a canonical, immune, and/or stromal metastatic molecular subtype.

71. The method of claim 70, wherein the classifier has been trained using a neural network machine learning process.

72. The method of claim 70 or 71, wherein the expression levels of all the genes listed on Table 1 are inputted into the classifier.

73. The method of any of claims 70-72, wherein no other expression levels are inputted into the classifier.
